(19) United States
(12) Patent Application Publication
(10) Pub. No.: US 2021/0085995 A1
(43) Pub. Date: Mar. 25, 2021

(54) APPARATUS AND METHOD FOR ADIPOSE TISSUE REDUCTION

(71) Applicants: MARK JULIAN, NEW PORT RICHEY, FL (US); MARGARET JULIAN, NEW PORT RICHEY, FL (US); CHAUNCEY WASHINGTON, CORONO, CA (US)

(72) Inventors: MARK JULIAN, NEW PORT RICHEY, FL (US); MARGARET JULIAN, NEW PORT RICHEY, FL (US); CHAUNCEY WASHINGTON, CORONO, CA (US)

(21) Appl. No.: 15/681,340

(22) Filed: Aug. 19, 2017

Related U.S. Application Data
(63) Continuation of application No. 62/494,003, filed on Aug. 22, 2016.

(51) Int. Cl. A61N 5/06

(52) U.S. Cl. CPC A61N 5/0616; A61N 2005/0632; A61N 2005/067; A61N 2005/0652; A61N 2005/0658; A61N 2005/0635

(57) ABSTRACT

A lipolysis apparatus and method of using a low power laser or light emitting diode (LED) light sources in conjunction with a mechanical vibrator to non-invasively irradiate a patient's skin to achieve reduction of adipose tissue, body shaping, and body contouring. The light source is in a range of red light with wavelength ranging from 620 nm to 629 nm and 10 MW to 50 MW in power output.

### REFERENCE TO RELATED APPLICATIONS

This application claims the benefit of U.S. Provisional Patent Application No. 62/494,003 filed Aug. 22, 2016, which is incorporated herein by reference.

### BACKGROUND OF THE INVENTION

### FIELD OF THE INVENTION

The present invention is in the technical field of medical devices, and more in the particularly, to non-invasive medical devices for fat removal, body shaping, and body contouring. The noninvasive device uses low-power laser irradiation, LED lightwaves and mechanical vibration.

### DESCRIPTION OF RELATED ART

There is a great demand for removal of excess adipose tissue, or fat, for health and aesthetic reasons. Cosmetic surgical procedure known as liposuction has been applied widely for targeted and local fat removal, body shaping, and body contouring. In a typical liposuction procedure, excess adipose tissue is suctioned from the body of a patient through one or more incisions. Since liposuction is an invasive surgical procedure, serious complications including deep vein thrombosis, organ perforation, bleeding, and infection may occur. The post-procedure recovery period is long and often accompanied by a great deal of inflammation, bruising and pain.

Non-invasive methods using electromagnetic energy, such as microwave, ultrasound or radio-frequency radiation, have been applied in fat reduction and body shaping. In particular, procedures and apparatuses have been developed to use low-power laser for non-invasive fat removal or reduction. For example, as described in U.S. Pat. No. 6,605,079B2, Neira, et al. applied one or more treatments of 635 nm laser energy externally to the patient to release at least a portion of the intracellular fat into the interstitial space through a transitory pore in the fat cell. Upon sufficient doses of this low level laser energy at 635 nm, the cell membrane is believed to be momentarily disrupted, releasing the intracellular fat into the interstitial space. Upon cessation of the energy application, the pore closes and the cell membrane returns to contiguity. The released fat is expected to be removed from the patient's body through one or more of the patient's normal bodily systems, including the lymphatic system.

Several non-invasive low-power laser systems have been developed in recent years to perform fat reduction and body contouring utilizing similar physical mechanism as mentioned above, including Zerona by Erchonia Corp, Strawberry by Laser Lipo Ltd, I-Lipo by Chromogenex, and a few others. These systems are being used commercially and have met commercial success in various degrees. However, the efficacy of this type of systems may be limited by the amount of fat released from cells undergoing membrane disruption due to laser irradiation, as well as by the amount of fat removal through normal bodily systems including the lymphatic system. An object of the present invention is to provide a non-invasive method of low-level laser therapy that increases the efficacy from removing fat from a patient.

### SUMMARY OF INVENTION

The present invention is a non-invasive lipolysis device that uses a low power laser or LED light source to irradiate skin. The efficacy of low-level laser of the device is enhanced by applying a mechanical vibration simultaneously to the surface of the treatment region with a vibrating motor. The mechanical vibration accelerates cellular fat release and lymphatic drainage, and therefore works simultaneously with the low-level light wave irradiation to remove fat, body shaping, and body contouring.

The present invention uses a low power laser or LED Red-light wave in the range of 620 nm to 660 nm. More particularly, it uses a beam of red light which may or may not be condensed through a lens or transparent window to obtain the same effect as lipolysis of adipose tissue exposed through incision using a conventional ultrasonic or low power laser. In addition, the lipolysis device includes a flexible head that can adhere to the body and vibration motors inside head. When the unit is used it is adhered to the body the light wave emits and vibration waves emit at the same time which readily discharges liquefied fat from an adipose cell and concentrated in a space between cell tissues through the groin area, where lymphatic vessels are abundant, and out of the body. Further the lipolysis device can stably contact human skin to break down fat and thus is convenient to use. Furthermore, the lipolysis device can break down subcutaneous fat by irradiating an abdominal region with a low power red light wave either through laser or LED light waves without skin damage or surgical operation, thereby effectively removing abdominal fat. Generally, an apparatus for generating a low power laser or LED includes a diode for emitting a red beam light wave having an output of 10 MW to 48 MW and a wavelength of 620 nm-629 nm, and a low power diode driver for arbitrarily adjusting the amount of beam emitted from the diode. Various apparatuses for obtaining a curative effect by irradiating spots on the body with a low power laser suitable for acupuncture or infected parts of the body. However, since the red light wave requires a separate radiation structure, there is no way of breaking down fat by non-invasively irradiating a human body. Meanwhile, in order to effectively treat obesity using a laser light wave, Neira, et al. discloses a new suction lipectomy capable of liquefying fat during suction lipectomy using a low power laser.

It is another aspect of the present invention to provide a belt, and so on, when the beam is disposed in the soft materials in an array, a red light wave capable of being output appropriately to non-invasively break down fat.

### BRIEF DESCRIPTION OF THE DRAWINGS

FIG. 1 is a diagram illustrating the main components of an embodiment of the lipolysis apparatus.

FIG. 2 is a diagram illustrating the main electronic components of the lipolysis apparatus.

FIG. 3(a) is a perspective front view of an embodiment of the light applicator.

FIG. 3(b) is a perspective back view of an embodiment of the light applicator.

FIG. 4 is a perspective view of the light applicator when “folded”

### REFERENCE NUMERALS IN THE DRAWINGS

For a more complete understanding of the present invention parts, reference is now made to the following descriptions:

100 Control unit.

102 Operator Input

103 Display panel.

104 Timer.

200 Treatment applicator.

201 Power inlet.

202 PCB control board.

203 Solid state relays.

204 Light source.

205 Vibration motor.

206 Treatment panel.

207 Motor Housing

208 Transparent window.

209 Applicator frame.

210 strap loop.

211 Flexible contact frame.

212 strap

300 Connection cable.

### DETAILED DESCRPTION OF THE INVENTION

The following detailed description includes preferred embodiments of the invention. The description is not to be taken in a limiting sense, but is made merely for the purpose of illustrating the general principles of the invention.

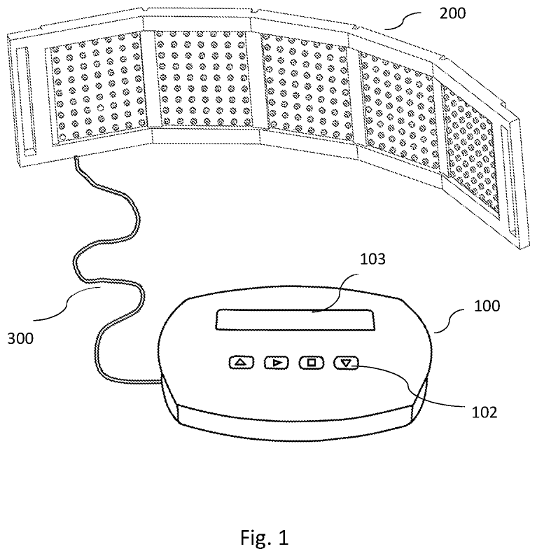

FIG. 1 is a diagram illustrating the main components of an embodiment of a lipolysis apparatus using a low power laser or LED light source. The portable apparatus comprises a control unit (100), a treatment applicator (200), and a connection cable (300) connecting the control unit (100) and the treatment applicator (200). On the control unit (100), there is an operator interface comprising input buttons (102) for entering setting parameters and a display panel (103) for displaying system and timer status.

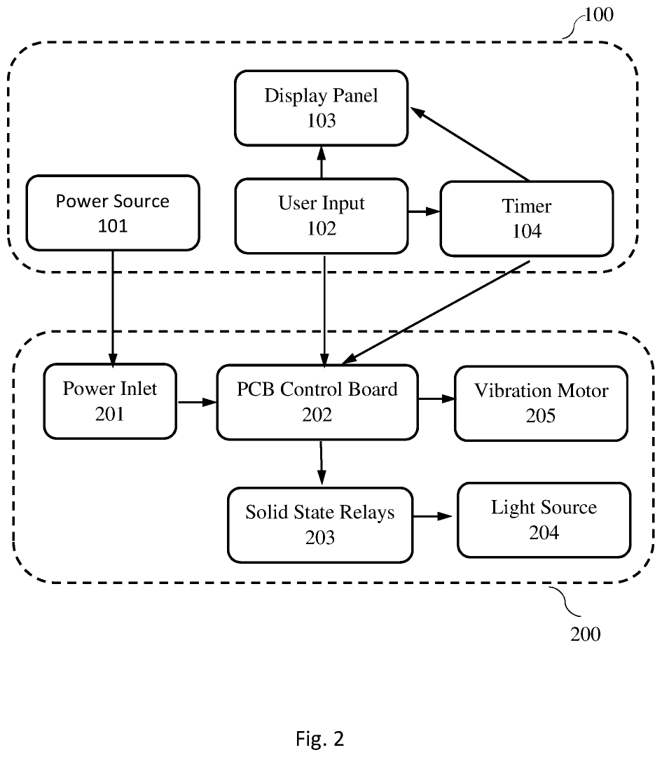

FIG. 2 further illustrates the internal components of the lipolysis apparatus. The control unit (100) houses a power source (101) which provides power to the treatment applicator (200) via the connection cable (300) and a power inlet (103). The operator input (102) component of the control unit (100) is connected to the PCB (printed circuit board) control board (202) of the treatment applicator (200) via the connection cable (300) as well. The operator input (102) also sets a timer (104). The display panel (103) displays system status, operator input, and the timer information. The PCB control board (202) is electronically connected to one or more solid state relays (203) that control that switch on and off the light sources (204). The PCB control board (202) is also electronically connected to and controls at least one vibration motor (205) that is/are housed in the treatment applicator (200) or attached to the treatment applicator (200).

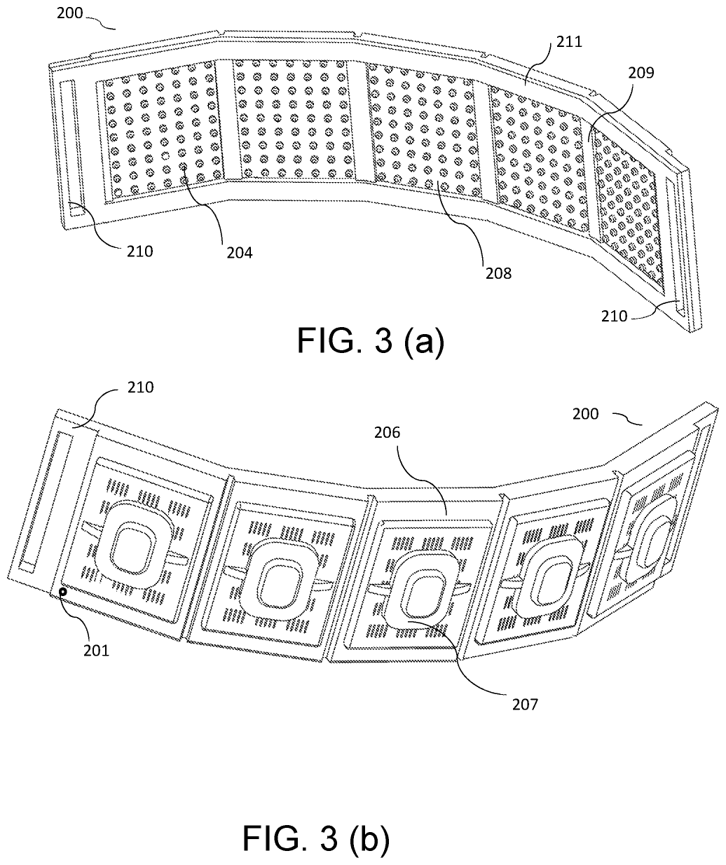

FIG. 3 shows the prospective front and back views of an embodiment of the treatment applicator (200). In this embodiment, the treatment applicator (200) comprises of multiple treatment panels (206). Each treatment panel (206) houses a PCB control board (202), a multiple of solid state relays (203), a multiple of light sources (204), such as light emitting diodes (LEDs), and a vibration motor (205) located in the motor housing (207) at the back of the treatment panel (206). The front of the treatment panel (206) comprises a transparent window (208) that allows light to pass through to reach the skin treatment area. The multiple of treatment panels (206) are arranged in an applicator frame (209) to form a belt-shaped applicator. Each end of the applicator frame comprises a strap loop (210). The applicator frame (209) also comprises a flexible contact frame (211) that is in touch with the patent skin during treatment. The flexible contact frame (211) is made of flexible materials, such as suitable synthetic polymer, and may be shaped to desirable contours to accommodate various sizes of the treatment area.

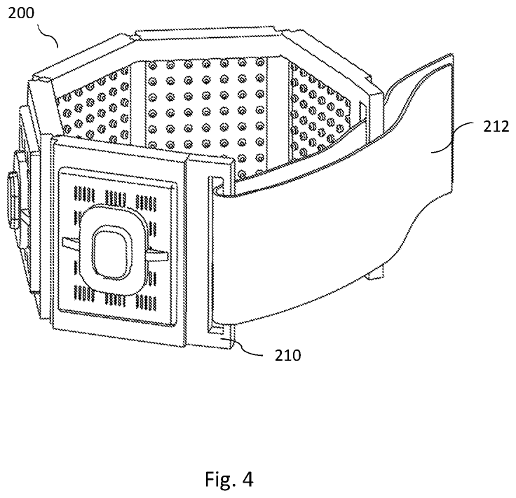

FIG. 4 shows the treatment applicator (200) configured in a bent contour to fit a treatment area, such as an abdomen and waist area. A section of strap (212) is fit through the strap loops (210) to fix the treatment applicator to the user's body. Velcro patches, clips, or other mechanisms can be used for fastening the strap.

Embodiments of the light source (204) can be light emitting diodes (LEDs) or laser sources. In some embodiments, their output power ranges from 10 MW to 50 MW. In other embodiments, they operate in the red wavelength range of 620 nm to 629 nm or in the range from 661 nm to 669 nm. There may be an additional heat dissipating plate located in the treatment panel (206) for dissipating heat generated from the light sources. The transparent windows (208) can be embodied as lenses to focus light to desirable intensity on the treatment area. The lipolysis device may be operated in a rhythm mode in which the vibration is repeatedly ran through a multitude of programs at predetermined intervals. The duration and the mode of operation can be setup by the operator using the operator input buttons (102). The applicator frame (209) may have grips formed at appropriate places on one or both sides thereof.

The foregoing description and accompanying drawings illustrate the principles, preferred or example embodiments, and modes of assembly and operation, of the invention; however, the invention is not, and shall not be construed as being exclusive or limited to the specific or particular embodiments set forth hereinabove.

What is claimed is:

1. A lipolysis apparatus for fat removal, body shaping, and body contouring, the lipolysis apparatus comprises:
a treatment applicator, wherein the said treatment applicator comprises at least one low power light source, the light source emits a wavelength in the range from about 620 nm to about 629 nm to a patient's treatment area, from said treatment applicator; and
at least one vibration motor, wherein the vibration motor is attached to the treatment applicator.

2. The lipolysis apparatus of claim 1, wherein the power output of each light source ranges from about 10 MW to about 50 MW.

3. The lipolysis apparatus of claim 2, wherein the light source is a light emitting diode (LED).

4. The lipolysis apparatus of claim 3, wherein the treatment applicator comprises a plurality of transparent windows or lenses disposed at one side surface, and a plurality of LED diodes that are located within the transparent windows or lenses.

5. The lipolysis apparatus of claim 4, further comprises at least one heat dissipating plate, wherein the said heat dissipating plate is mechanically connected to the said treatment applicator.

6. The lipolysis apparatus of claim 1, wherein the treatment applicator can be bent to conform to the contours of the treatment area.

7. The lipolysis apparatus of claim 6, wherein the treatment applicator comprises:
(a) a flexible frame that can be bent up to 360 degrees; and
(b) a multiple of treatment panels, wherein each treatment panel comprises of a multiple of LED light sources, a transparent window that allows light to pass through to reach the skin treatment area, and a vibration motor.

8. The lipolysis apparatus of claim 7, further comprises a fastener that will attach to a patient's treatment area.

9. The lipolysis apparatus of claim 8, wherein the fastener comprises of at least one strap loop on the flexible frame and at least one strap that can be fastened to the user.

10. The lipolysis apparatus of claim 1, further comprising a control unit, wherein the control unit has a display screen and an operator input interface.

11. The lipolysis apparatus of claim 10, further comprising a timer that controls the time duration of the treatment, wherein the time duration of the timer can be set by using the operator input interface.

12. The lipolysis apparatus of claim 10, wherein the operator input interface comprises a button to start the treatment and a button to stop the treatment.

13. The lipolysis apparatus for fat removal, body shaping, and body contouring, the lipolysis apparatus of claim 1, wherein the light source is a low power laser.

14. A lipolysis apparatus for fat removal, body shaping, and body contouring, the lipolysis apparatus comprises:
a treatment applicator, wherein the said treatment applicator comprises at least one low power light source, the light source emits a wavelength in the range from about 661 nm to about 669 nm to a patient's treatment area, from said treatment applicator; and
at least one vibration motor, wherein the vibration motor is attached to the treatment applicator.

15. A lipolysis apparatus for fat removal, body shaping, and body contouring, the lipolysis apparatus of claim 14, wherein the light source is a low power laser.

16. A method for removing or reducing adipose tissue from the body of a patient, comprising:
a) applying laser or LED light to the adipose tissue externally; and
b) simultaneously applying mechanical vibration to the area of the body where the laser energy is applied.

17. The method for removing or reducing adipose tissue from the body of a patient of claim 16, wherein an array of laser or LED light sources are used to irradiate the treatment area, and the peak wavelength range is approximately between 620 nm to approximately 629 nm, and wherein the time duration to apply the light irradiation and vibration treatment ranges from about 5 to about 15 minutes.
